(19) United States
(12) Patent Application Publication
(10) Pub. No.: US 2015/0265178 A1
(43) Pub. Date: Sep. 24, 2015

(54) METHOD AND APPARATUS FOR DETERMINING CHARACTERISTICS OF CEREBRAL ISCHEMIA BASED ON MAGNETIC RESONANCE DIFFUSION WEIGHTED IMAGING

(71) Applicant: SHENZHEN INSTITUTES OF ADVANCED TECHNOLOGY, Shenzhen, Guangdong (CN)

(72) Inventor: Qingmao HU, Nanshan Shenzhen, Guangdong (CN)

(21) Appl. No.: 14/439,156

(22) PCT Filed: Sep. 25, 2014
(86) PCT No.: PCT/CN2014/087406

§ 371 Date: Apr. 28, 2015

(30) Foreign Application Priority Data
Sep. 26, 2013 (CN) 201310446792.0

(51) Int. Cl. A61B 5/055; A61B 5/026; G01R 33/563

(52) U.S. Cl. CPC A61B 5/055; G01R 33/56341; A61B 5/0263; A61B 2576/00

(57) ABSTRACT

The present invention discloses a method and apparatus for determining characteristics of cerebral ischemia based on magnetic resonance diffusion weighted imaging, so as to provide a more objective basis for determining whether an acute cerebral ischemia patient should be treated with thrombolysis. The method comprises: determining a cerebral ischemia region of a patient based on magnetic resonance diffusion weighted imaging of the patient, wherein the cerebral ischemia region comprises a core region and a transition region; determining an apparent diffusion coefficient ADC characteristic parameter ADCᵣ in a region with high DWI values in the magnetic resonance diffusion weighted imaging according to diffusion weighted image DWI values in the core region and transition region; and judging whether ADC values in the region with high DWI values and the region with high DWI values are mismatched according to the ADC characteristic parameter ADCᵣ. The method according to embodiments of the present invention provides a more scientific and objective basis for making a decision on whether the acute cerebral ischemia patient should be treated with thrombolysis, thereby improving a cure rate of the cerebral ischemia patient.

### TECHNICAL FIELD

The present invention relates to the biomedical imaging field, and in particular to a method and apparatus for determining characteristics of cerebral ischemia based on magnetic resonance diffusion weighted imaging.

### BACKGROUND

In China, the morbidity of cerebrovascular diseases increases year by year. In recent years, the epidemiological survey results show that the cerebrovascular disease ranks second only to the malignant tumor as a cause of death in China. The cerebrovascular disease has a high disability rate, which causes serious damage to the health and survival quality of human beings. Wherein, the ischemic cerebral apoplexy (cerebral infarction) accounts for more than 70% of the cerebrovascular diseases. Therefore, strengthening the study of the cerebral infarction is particularly important.

For the ischemic cerebral apoplexy, the guidelines of all countries recommend that it is preferred to select intravenous administration of recombinant tissue plasminogen activator (rtPA) for thrombolysis treatment at the onset. Intravenous administration of recombinant tissue plasminogen activator for thrombolysis is proved to be an effective means for the treatment of ischemic cerebral apoplexy. However, the thrombolysis treatment is particularly prone to serious complications such as bleeding, and must be used strictly according to the characters of brain ischemia of patients. However, how to clearly learn the pathological state such as the characteristics of brain ischemia of patients has long been a problem difficult to resolve in medicine.

An existing method for thrombolysis treatment of patient of cerebral ischemia in super acute period is mainly based on a time window, that is, it stipulates only when onset time of the patient is less than 4.5 hours and the patient does not bleed or have bleeding symptom, the thrombolysis is allowed. However, a majority of ischemic cerebral apoplexy patients cannot see a doctor within 4.5 hours, resulting in the problem of under-treatment; some patients have a good prognosis after 4.5 hours even without thrombolysis, and it is overtreatment if the thrombolysis is applied.

It can be seen that although the existing method for guiding the thrombolysis treatment of patients of cerebral ischemia is based on the treatment principle consistent with the provisions of the guidelines such as the time window (4.5 hours), existence of a cerebral ischemia region (magnetic resonance DWI representation) but without a bleeding region (represented by using X-ray computed tomography image CT), the patients who meet the foregoing conditions may not necessarily benefit from thrombolysis, and many of the cerebral ischemia patients who do not have cerebral hemorrhage in 4.5 hours after the onset may benefit from thrombolysis. In other words, the existing method for guiding thrombolysis for acute cerebral ischemia patients is not based on an accurate grasp of characteristics of cerebral ischemia of patients; therefore, the existing method is still not satisfactory.

### SUMMARY

Embodiments of the present invention provide a method and apparatus for determining characteristics of cerebral ischemia based on magnetic resonance diffusion weighted imaging, so as to provide a more objective basis for determining whether an acute cerebral ischemia patient should be treated with thrombolysis.

An embodiment of the present invention provides a method for determining characteristics of cerebral ischemia based on magnetic resonance diffusion weighted imaging, wherein the method comprises:

determining a cerebral ischemia region of a patient based on magnetic resonance diffusion weighted imaging of the patient, wherein the cerebral ischemia region comprises a core region and a transition region;

determining an apparent diffusion coefficient ADC characteristic parameter ADCᵣ in a region with high DWI values in the magnetic resonance diffusion weighted imaging according to diffusion weighted image DWI values in the core region and transition region; and

judging whether ADC values in the region with high DWI values and the region with high DWI values are mismatched according to the ADC characteristic parameter ADCᵣ.

Another embodiment of the present invention provides an apparatus for determining characteristics of cerebral ischemia based on magnetic resonance diffusion weighted imaging, wherein the apparatus comprises:

a cerebral ischemia region determining module, configured to determine a cerebral ischemia region of a patient based on magnetic resonance diffusion weighted imaging DWI of the patient, wherein the cerebral ischemia region comprises a core region and a transition region;

a characteristic parameter determining module, configured to determine an apparent diffusion coefficient ADC characteristic parameter ADCᵣ in a region with high DWI values in the magnetic resonance diffusion weighted imaging according to DWI values in the core region and transition region; and

a judging module, configured to judge whether ADC values in the region with high DWI values and the region with high DWI values are mismatched according to the ADC characteristic parameter ADCᵣ.

It can be seen from the foregoing embodiments of the present invention that, the determining of the apparent diffusion coefficient ADC characteristic parameter ADCᵣ in the region with high DWI values in the magnetic resonance diffusion weighted imaging is based on the ADC values in the cerebral ischemia region, that is, the core region and the transition region, and whether the cerebral ischemia patient should be treated with thrombolysis is finally determined based on whether the ADC values in the region with high DWI values and the region with high DWI values are mismatched. It can be seen that the method according to the embodiment of the present invention does not simply use a pure time window as the main decision-making basis, but establishes joint characteristics through conjoint analysis of magnetic resonance ADC and DWI. Compared with the method for treating cerebral ischemia based on the time window (for example, patients with cerebral ischemia within 4.5 hours are treated with thrombolysis and patients with cerebral ischemia greater than 4.5 hours are not treated with thrombolysis). The method according to the embodiment of the present invention provides a more scientific and objective basis for making a decision on whether an acute cerebral ischemia patients should be treated with thrombolysis, thereby improving a cure rate of the cerebral ischemia patient.

### BRIEF DESCRIPTION OF THE DRAWINGS

FIG. 1 is a schematic basic flow chart of a method for determining characteristic of cerebral ischemia based on magnetic resonance diffusion weighted imaging according to an embodiment of the present invention;

FIG. 2 is a schematic logical structure diagram of an apparatus for determining characteristics of cerebral ischemia based on magnetic resonance diffusion weighted imaging according to an embodiment of the present invention;

FIG. 3 is a schematic logical structure diagram of an apparatus for determining characteristics of cerebral ischemia based on magnetic resonance diffusion weighted imaging according to another embodiment of the present invention;

FIG. 4 is a schematic logical structure diagram of an apparatus for determining characteristics of cerebral ischemia based on magnetic resonance diffusion weighted imaging according to another embodiment of the present invention;

FIG. 5 is a schematic logical structure diagram of an apparatus for determining characteristics of cerebral ischemia based on magnetic resonance diffusion weighted imaging according to another embodiment of the present invention; and

FIG. 6 is a schematic logical structure diagram of an apparatus for determining characteristics of cerebral ischemia based on magnetic resonance diffusion weighted imaging according to another embodiment of the present invention.

### DESCRIPTION OF EMBODIMENTS

An embodiment of the present invention provides a method for determining characteristics of cerebral ischemia based on magnetic resonance diffusion weighted, comprising: determining a cerebral ischemia region of a patient based on magnetic resonance diffusion weighted imaging of the patient, wherein the cerebral ischemia region comprises a core region and a transition region; determining an apparent diffusion coefficient ADC characteristic parameter ADCᵣ in a region with high DWI values in the magnetic resonance diffusion weighted imaging according to diffusion weighted image DWI values in the core region and transition region; and judging whether ADC values in the region with high DWI values and the region with high DWI values are mismatched according to the ADC characteristic parameter ADCᵣ. An embodiment of the present invention also provides a corresponding apparatus for determining characteristics of cerebral ischemia based on magnetic resonance diffusion weighted imaging. They are described in detail and respectively in the following.

Reference may be made to FIG. 1 for a basic flow of a method for determining characteristics of cerebral ischemia based on magnetic resonance diffusion weighted imaging according to an embodiment of the present invention, wherein the method mainly includes the following steps S101 to S103:

S101. Determine a cerebral ischemia region of a patient based on magnetic resonance diffusion weighted imaging of the patient, wherein the cerebral ischemia region comprises a core region and a transition region.

In this embodiments of the present invention, the magnetic resonance diffusion weighted imaging of the patient comprises an isotropic diffusion weighted image (DWI) with a high diffusion sensitivity factor b, a T2 weighted image with b=0, and an apparent diffusion coefficient (ADC) map obtained by calculating the DWI and the T2 weighted image. As an embodiment of the present invention, the determining a cerebral ischemia region of a patient based on magnetic resonance diffusion weighted imaging of the patient comprises: calculating the ADC values of voxels in the magnetic resonance diffusion weighted imaging, determining a region of which the ADC values of voxels in the magnetic resonance diffusion weighted imaging are less than D₁×ADC_{ref} as the core region; and determining a region of which the ADC values of voxels in the magnetic resonance diffusion weighted imaging are in the range of [D₁×ADC_{ref}, D₂×ADC_{ref}] and that is spatially adjacent to the core region as the transition region, wherein ADC_{ref} is the ADC value of normal brain tissues, and is the value that has the highest frequency of occurrence in the ADC image, D₁ is a constant in the range of [0.6, 0.7], and D₂ is a constant in the range of [0.8, 0.9]. Specifically, determining the cerebral ischemia region comprises: based on the obtained T2 weighted image, calculating and distinguishing the brain tissue and non-brain tissue to obtain the brain tissue image without the non-brain tissue brain(x, y, z), for positioning and obtaining relevant parameters in the ADC image; according to the ADC threshold thADC2 of the transition region obtained by calculation, performing a binarization of hypointense signal constraint on the ADC image corresponding to the brain tissue image, so as to obtain the binary image B_ADC (x, y, z); estimating the core region and transition region according to the binary image and the core region obtained by calculation, and performing hyperintense signal constraint processing on the core region according to the DWI hyperintense signal characteristics of the core region obtained by calculation, so as to obtain the core region and the transition region.

S102. Determine an apparent diffusion coefficient ADC characteristic parameter ADCᵣ in a region with high DWI values in the magnetic resonance diffusion weighted imaging according to diffusion weighted image DWI values in the core region and transition region.

The region with high DWI values in the magnetic resonance diffusion weighted imaging corresponds to severe cerebral ischemia. In this embodiment of the present invention, an estimation of drawing the region with high DWI and using the region with high DWI as severe cerebral ischemia (cerebral infarction) region may be firstly achieved by obtaining the threshold Th₁ which is used to determine the region with high DWI. To ensure that the DWI values in a certain region in the magnetic resonance diffusion weighted imaging are high, the threshold Th₁ may be determined by using a more conservative method. Specifically, the determining an apparent diffusion coefficient ADC characteristic parameter ADCᵣ in a region with high DWI values in the magnetic resonance diffusion weighted imaging according to diffusion weighted image DWI values in the core region and transition region comprises: calculating an average DWI gray scale value DWI_{avg} and a maximum DWI value DWIₘₐₓ according to the DWI values in the core region; determining a region that consists of voxels with the DWI values being not less than Th₁ in the core region and transition region as the region with high DWI values in the magnetic resonance diffusion weighted imaging, wherein Th₁ is a preset value or a constant in the range of (DWI_{avg}+DWIₘₐₓ)/2, 1.1×(DWI_{avg}+DWIₘₐₓ)/2]; and calculating the ADC values in the region with high DWI values, and using a ratio of the voxels in the region with high DWI values and whose ADC values are not less than C₁×ADC_{ref} to all voxels in the region with high DWI values as the ADC characteristic parameter ADCr in the region with high DWI values in the magnetic resonance diffusion weighted imaging, wherein the ratio is used to represent a size or ratio of the ADC based region in which the ischemia is not serious, C1 is a constant in the range of [0.6, 0.7], and the definition of ADC_{ref} is the same as that described in the foregoing embodiment, that is, ADC_{ref} is the ADC values of normal brain tissues.

S103. Judge whether ADC values in the region with high DWI values and the region with high DWI values are mismatched according to the ADC characteristic parameter ADCᵣ.

The region with high DWI values in the magnetic resonance diffusion weighted imaging corresponds to severe cerebral ischemia, and the corresponding ADC should present a hypointense signal. Once the DWI and the ADC are mismatched, there would be more voxels with higher ADC (corresponding to the non-serious ischemia based on the ADC) in the region with high DWI values. One way is use the ADCᵣ determined in the foregoing embodiment to judge whether ADC and DWI are mismatched. In other words, if ADCᵣ is large enough, it indicates that ADC and DWI are mismatched. Therefore, a threshold for ADCᵣ needs to be determined; if a patient's ADCᵣ is not less than the threshold, it is judged that ADC and DWI are mismatched. The threshold may be obtained by experience or learning. A method for obtaining the threshold by learning is as follows: assume that the magnetic resonance diffusion weighted imaging (comprising DWI and ADC image) of N patients with onset time within nine hours or longer has been obtained; therefore, the region with high DWI values in the magnetic resonance diffusion weighted imaging and the ratio ADCᵣ that ADC is not less than C₁×ADC_{ref} in the region can be calculated for each patient; whether the N patients are treated with thrombolysis and the prognoses of the patients are good or bad are learned, such that the sensitivity and specificity on whether N patients are treated with thrombolysis can be determined according to the threshold of the ADCᵣ. Specifically, judging whether the ADC values in the region with high DWI values and the region with high DWI values are mismatched according to the ADC characteristic parameter ADCᵣ comprises steps S1031 and S1032.

S1031. Determine a threshold Thresh_{ADC} which is used to judge whether the ADC values in the region with high DWI values and the region with high DWI values are mismatched according to obtained statistical data on whether N patients are treated with thrombolysis and whether prognoses of the patients are good or bad, wherein N is a natural number greater than 1.

In the clinical medicine, the patient with cerebral ischemia is presented by: true positive (TP) when the patient has good prognosis after thrombolysis and has bad prognosis without thrombolysis and ADCᵣ≧Thresh_{ADC}; true negative (TN) when the patient has bad prognosis after thrombolysis and has good prognosis without thrombolysis and ADCᵣ<Thresh_{ADC}; false positive (FP) when the patient has bad prognosis after thrombolysis and has good prognosis without thrombolysis and ADCᵣ≧Thresh_{ADC}; and false negative (FN) when the patient has good prognosis after thrombolysis and has bad prognosis without thrombolysis and when ADCᵣ<Thresh_{ADC}. In this embodiment of the present invention, the determining a threshold Thresh_{ADC} which is used to judge whether the ADC values in the region with high DWI values and the region with high DWI values are mismatched according to the obtained statistical data on whether the N patients are treated with thrombolysis and whether the prognoses of the patients are good or bad may be achieved in the following way: obtaining a value S_{TP}/(S_{TP}+S_{FN}) indicative of sensitivity and a value S_{TN}/(S_{FP}+S_{TN}) indicative of specificity by performing statistics, among the N patients, on a sum S_{TP} of patients who have good prognosis after thrombolysis and who have bad prognosis without thrombolysis when ADCᵣ is greater than or equal to the threshold Thresh1 to be determined, a sum S_{TN} of patients who have bad prognosis after thrombolysis and who have good prognosis without thrombolysis when ADCᵣ is less than the threshold Thresh1 to be determined, a sum S_{FP} of patients who have bad prognosis after thrombolysis and who have good prognosis without thrombolysis when ADCᵣ is greater than or equal to the threshold Thresh1 to be determined, and a sum S_{FN} of patients who have good prognosis after thrombolysis and who have bad prognosis without thrombolysis when ADCᵣ is less than the threshold Thresh1 to be determined; and calculating a value of the threshold Thresh1 to be determined that makes S_{TP}/(S_{TP}+S_{FN})+S_{TN}/(S_{FP}+S_{TN}) reach a maximum value, and using the value of the threshold Thresh1 to be determined that makes S_{TP}/(S_{TP}+S_{FN})+S_{TN}/(S_{FP}+S_{TN}) reach the maximum value as the threshold Thresh_{ADC} which is used to judge whether the ADC values in the region with high DWI values and the region with high DWI values are mismatched. In other words, it is assumed that the value of the threshold Thresh₁ to be determined that makes S_{TP}/(S_{TP}+S_{FN})+S_{TN}/(S_{FP}+S_{TN}) reach the maximum value is Threshₘₐₓ, and Thresh_{DWI}=Threshₘₐₓ.

S1032. If the ADCᵣ in the region with high DWI values in the magnetic resonance diffusion weighted imaging is not less than the threshold Thresh_{ADC}, judge that the ADC values in the region with high DWI values and the region with high DWI values are mismatched. For those with mismatched ADC values, it is proposed that the medical staff should treat such patients with thrombolysis, to reduce mortality and disability rate.

It can be seen from the method for determining characteristics of cerebral ischemia based on magnetic resonance diffusion weighted imaging according to the foregoing embodiment of the present invention that, the determining of the apparent diffusion coefficient ADC characteristic parameter ADCᵣ in the region with high DWI values in the magnetic resonance diffusion weighted imaging is based on the ADC values in the cerebral ischemia region, that is, the core region and the transition region, and whether the cerebral ischemia patient should be treated with thrombolysis is finally determined based on whether the ADC values in the region with high DWI values and the region with high DWI values are mismatched. It can be seen that the method according to the embodiment of the present invention does not simply use a pure time window as the main decision-making basis, but establishes joint characteristics through conjoint analysis of magnetic resonance ADC and DWI. Compared with the method for treating cerebral ischemia based on the time window (for example, patients with cerebral ischemia within 4.5 hours are treated with thrombolysis and patients with cerebral ischemia greater than 4.5 hours are not treated with thrombolysis). The method according to this embodiment of the present invention provides a more scientific and objective basis for making a decision on whether an acute cerebral ischemia patient should be treated with thrombolysis, thereby improving a cure rate of the cerebral ischemia patient.

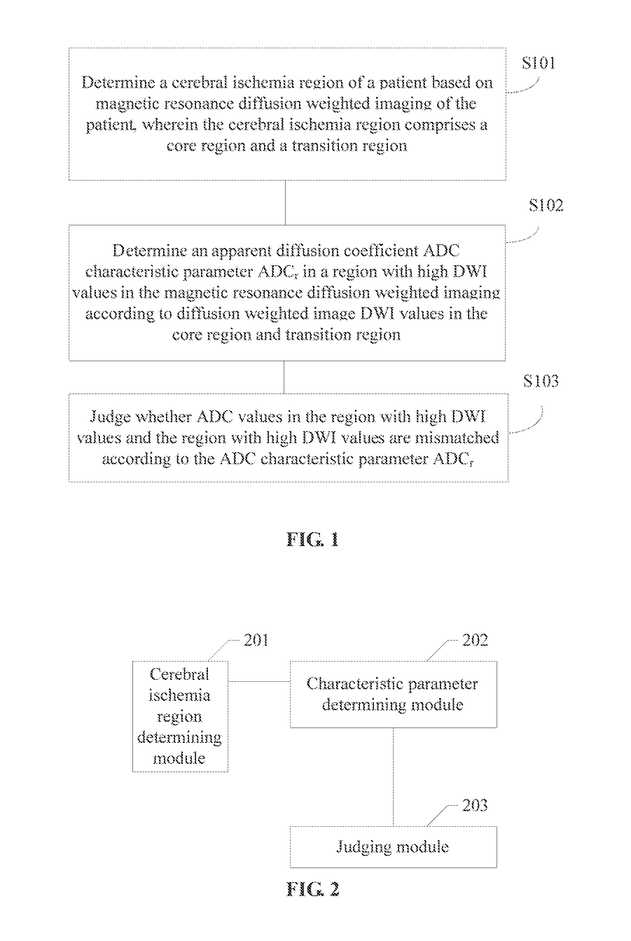

The following provides a description of an apparatus for determining characteristic of cerebral ischemia based on magnetic resonance diffusion weighted imaging according to an embodiment of the present invention, which is configured to execute the method for determining characteristics of cerebral ischemia based on magnetic resonance diffusion weighted imaging. For a basic logical structure of the apparatus, reference may be made to FIG. 2. For illustration purposes, the apparatus for determining characteristics of cerebral ischemia based on magnetic resonance diffusion weighted imaging only show the parts relative to the embodiment of the present invention in FIG. 2, and mainly comprises a cerebral ischemia region determining module 201, a characteristic parameter determining module 202, and a judging module 203. Each module is described in detail as follows:

The cerebral ischemia region determining module 201 is configured to determine a cerebral ischemia region of a patient based on magnetic resonance diffusion weighted imaging of the patient, wherein the cerebral ischemia region comprises a core region and a transition region.

The characteristic parameter determining module 202 is configured to determine an apparent diffusion coefficient ADC characteristic parameter ADCᵣ in a region with high DWI values in the magnetic resonance diffusion weighted imaging according to DWI values in the core region and transition region.

The judging module 203 is configured to judge whether ADC values in the region with high DWI values and the region with high DWI values are mismatched according to the ADC characteristic parameter ADCᵣ.

It should be noted that, in the foregoing implementing manners of the apparatus for determining characteristic of cerebral ischemia based on magnetic resonance diffusion weighted imaging illustrated in FIG. 2, division of functional modules is only an example for illustration, while in a practical application, the foregoing functions can be assigned to be completed by different functional modules according to a configuration requirement of corresponding hardware or out of consideration for facilitating implementation of software, that is, an internal structure of the apparatus for determining characteristic of cerebral ischemia based on magnetic resonance diffusion weighted imaging is divided into different functional modules to complete all or part of the foregoing functions. Moreover, in a practical application, corresponding functional modules in the embodiments can be implemented by corresponding hardware, and can also be implemented by corresponding hardware that executes corresponding software. For example, the foregoing cerebral ischemia region determining module can be hardware that determines a cerebral ischemia region of a patient based on magnetic resonance diffusion weighted imaging of the patient, such as a cerebral ischemia region determining apparatus, and can also be a general processor or another hardware device capable of executing a corresponding computer program to implement the foregoing functions or a general receiving apparatus capable of executing the foregoing functions; the foregoing characteristic parameter determining module can be hardware that determines an apparent diffusion coefficient ADC characteristic parameter ADCᵣ in the region with high DWI values in the magnetic resonance diffusion weighted imaging according to the DWI values in the core region and transition region, such as a characteristic parameter determining apparatus, and can also be a general processor or another hardware device capable of executing a corresponding computer program to implement the foregoing functions or a general receiving apparatus capable of executing the foregoing functions (each embodiment provided by the present specification can use the foregoing principle).

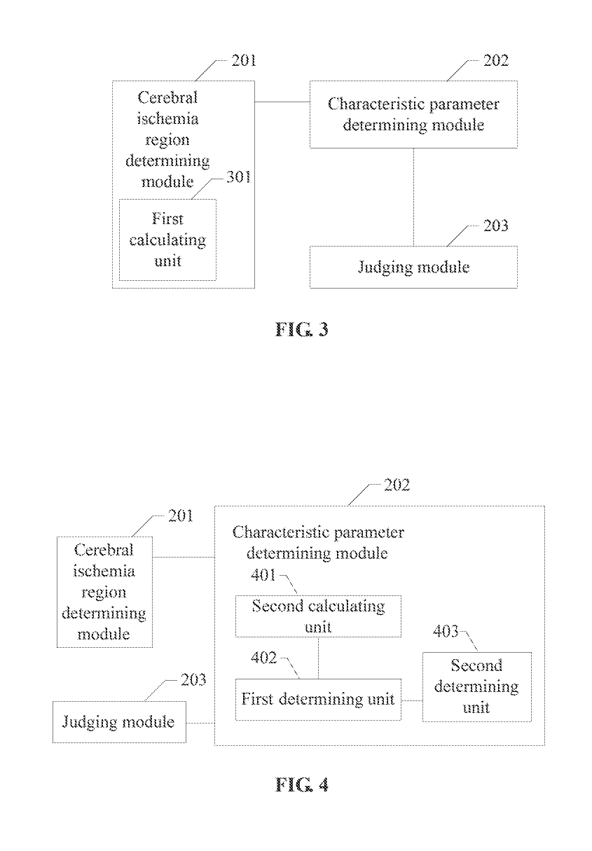

In the apparatus for determining characteristics of cerebral ischemia based on magnetic resonance diffusion weighted imaging illustrated in FIG. 2, the cerebral ischemia region determining module 201 may comprise a first calculating unit 301. Referring to FIG. 3, another embodiment of the present invention provides an apparatus for determining characteristic of cerebral ischemia based on magnetic resonance diffusion weighted imaging. The first calculating unit 301 is configured to calculate the ADC values of voxels in the magnetic resonance diffusion weighted imaging, determine a region of which the ADC values of voxels in the magnetic resonance diffusion weighted imaging are less than D₁×ADC_{ref} as the core region, and determine a region of which the ADC values of voxels in the magnetic resonance diffusion weighted imaging are in the range of [D₁×ADC_{ref}, D₂×ADC_{ref}] and that is spatially adjacent to the core region as the transition region, wherein ADC_{ref} is the ADC values of normal brain tissues, D₁ is a constant in the range of [0.6, 0.7], and D₂ is a constant in the range of [0.8, 0.9].

In the apparatus for determining characteristics of cerebral ischemia based on magnetic resonance diffusion weighted imaging shown in FIG. 2, the characteristic parameter determining module 202 may comprise a second calculating unit 401, a first determining unit 402 and a second determining unit 403, referring to the apparatus for determining characteristics of cerebral ischemia based on magnetic resonance diffusion weighted imaging according to another embodiment of the present invention illustrated in FIG. 4.

The second calculating unit 401 is configured to calculate an average DWI gray scale value DWI_{avg} and a maximum DWI value DWIₘₐₓ according to the DWI values in the core region.

The first determining unit 402 is configured to determine a region that consists of the voxels that the DWI values being not less than Th₁ in the core region and transition region as the region with high DWI values in the magnetic resonance diffusion weighted imaging, wherein Th₁ is a preset value or a constant in the range of (DWI_{avg}+DWIₘₐₓ)/2, 1.1×(DWI_{avg}+DWIₘₐₓ)/2].

The second determining unit 403 is configured to calculate the ADC values in the region with high DWI values, and use a ratio of the voxels in the region with high DWI values and whose ADC values are not less than C₁×ADC_{ref} to all voxels in the region with high DWI values as the ADC characteristic parameter ADCᵣ in the region with high DWI values in the magnetic resonance diffusion weighted imaging, wherein C is a constant in the range of [0.6, 0.7], and ADC_{ref} is the ADC values of normal brain tissues.

In the apparatus for determining characteristic of cerebral ischemia based on magnetic resonance diffusion weighted imaging illustrated in FIG. 2, the judging module 203 may comprise a third determining submodule 501 and a first judging submodule 502, referring to the apparatus for determining characteristics of cerebral ischemia based on magnetic resonance diffusion weighted imaging according to another embodiment of the present invention illustrated in FIG. 5.

The third determining submodule 501 is configured to determine a threshold Thresh_{ADC} which is used to judge whether the ADC values in the region with high DWI values and the region with high DWI values are mismatched according to obtained statistical data on whether N patients are treated with thrombolysis and whether prognoses of the patients are good or bad, wherein N is a natural number greater than 1.

The first judging submodule 502 is configured to, if the ADCᵣ in the region with high DWI values in the magnetic resonance diffusion weighted imaging is not less than the threshold Thresh_{ADC}, judge that the ADC values in the region with high DWI values and the region with high DWI values are mismatched.

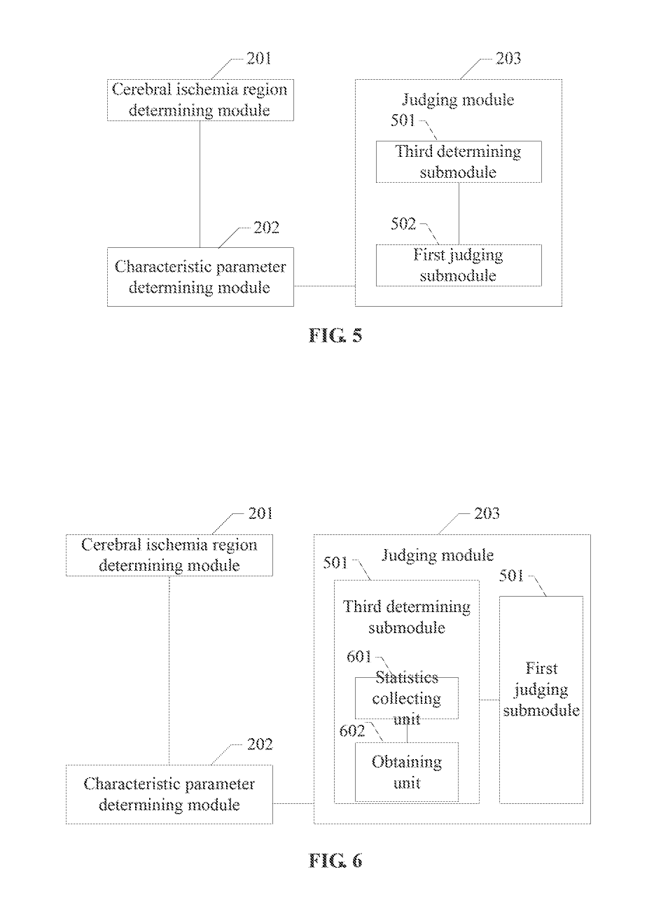

In the apparatus for determining characteristic of cerebral ischemia based on magnetic resonance diffusion weighted imaging illustrated in FIG. 5, the third determining module 501 may comprise a statistics collecting unit 601 and an obtaining unit 602, referring to the apparatus for determining characteristic of cerebral ischemia based on magnetic resonance diffusion weighted imaging according to another embodiment of the present invention illustrated in FIG. 6.

The statistics collecting unit 601 is configured to obtain a value S_{TP}/(S_{TP}+S_{FN}) indicative of sensitivity and a value S_{TN}/(S_{FP}+S_{TN}) indicative of specificity by performing statistics, among the N patients, on a sum S_{TP} of patients who have good prognosis after thrombolysis and who have bad prognosis without thrombolysis when ADCᵣ is greater than or equal to the threshold Thresh1 to be determined, a sum S_{TN} of patients who have bad prognosis after thrombolysis and who have good prognosis without thrombolysis when ADCᵣ is less than the threshold Thresh1 to be determined, a sum S_{FP} of patients who have bad prognosis after thrombolysis and who have good prognosis without thrombolysis s when ADCᵣ is greater than or equal to the threshold Thresh1 to be determined, and a sum S_{FN} of patients who have good prognosis after thrombolysis and who have bad prognosis without thrombolysis when ADCᵣ is less than the threshold Thresh1 to be determined.

The obtaining unit 602 is configured to calculate a value of the threshold Thresh1 to be determined that makes S_{TP}/(S_{TP}+S_{FN})+S_{TN}/(S_{FP}+S_{TN}) reach a maximum value, and use the value of the threshold Thresh1 to be determined that makes S_{TP}/(S_{TP}+S_{FN})+S_{TN}/(S_{FP}+S_{TN}) reach the maximum value as the threshold Thresh_{ADC} which is used to judge whether the ADC values in the region with high DWI values and the region with high DWI values are mismatched.

Another embodiment of the present invention provides a magnetic resonance diffusion weighted imaging processing device, comprising an input apparatus, an output apparatus, a memory, and a processor, wherein the processor executes the following steps: determining a cerebral ischemia region of a patient based on magnetic resonance diffusion weighted imaging of the patient, wherein the cerebral ischemia region comprises a core region and a transition region; determining an apparent diffusion coefficient ADC characteristic parameter ADCᵣ in a region with high DWI values in the magnetic resonance diffusion weighted imaging according to diffusion weighted image DWI values in the core region and transition region; and judging whether ADC values in the region with high DWI values and the region with high DWI values are mismatched according to the ADC characteristic parameter ADCᵣ.

An embodiment of the present invention provides a magnetic resonance diffusion weighted imaging processing device, comprising an input apparatus, an output apparatus, a memory, a processor, and one or more than one program, wherein the one or more than one program is stored in the memory, and the one or more than one program is executed by one or more than one processor to execute the instructions for performing the following operations:

determining a cerebral ischemia region of a patient based on magnetic resonance diffusion weighted imaging of the patient, wherein the cerebral ischemia region comprises a core region and a transition region;

determining an apparent diffusion coefficient ADC characteristic parameter ADCᵣ in a region with high DWI values in the magnetic resonance diffusion weighted imaging according to diffusion weighted image DWI values in the core region and transition region; and

judging whether ADC values in the region with high DWI values and the region with high DWI values are mismatched according to the ADC characteristic parameter ADCᵣ.

Assuming the foregoing is a first possible implementation manner, in a second possible implementation manner based on the first possible implementation manner, the memory further comprises instructions for performing the following operations:

calculating the ADC values of voxels in the magnetic resonance diffusion weighted imaging; determining a region of which the ADC values of voxels in the magnetic resonance diffusion weighted imaging are less than D₁×ADC_{ref} as the core region; and determining a region of which the ADC values of voxels in the magnetic resonance diffusion weighted imaging are in the range of [D₁×ADC_{ref}, D₂×ADC_{ref}] and that is spatially adjacent to the core region as the transition region, wherein ADC_{ref} is the ADC value of normal brain tissues, D₁ is a constant in the range of [0.6, 0.7], and D₂ is a constant in the range of [0.8, 0.9].

Assuming the foregoing is the second possible implementation manner, in a third possible implementation manner based on the first possible implementation manner, the memory further comprises instructions for performing the following operations:

calculating an average DWI gray scale value DWI_{avg} and a maximum DWI value DWIₘₐₓ according to the DWI values in the core region;

determining a region that consists of voxels with the DWI values being not less than Th₁ in the core region and transition region as the region with high DWI values in the magnetic resonance diffusion weighted imaging, wherein Th₁ is a preset value or a constant in the range of (DWI_{avg}+DWIₘₐₓ)/2, 1.1×(DWI_{avg}+DWIₘₐₓ)/2]; and

calculating the ADC values in the region with high DWI values, and using a ratio of the voxels in the region with high DWI values and whose ADC values are not less than C₁×ADC_{ref} to all voxels in the region with high DWI values as the ADC characteristic parameter ADCᵣ in the region with high DWI values in the magnetic resonance diffusion weighted imaging, wherein C1 is a constant in the range of [0.6, 0.7], and ADC_{ref} is the ADC values of normal brain tissues.

Assuming the foregoing is the third possible implementation manner, in a fourth possible implementation manner based on the first possible implementation manner, the memory further comprises instructions for performing the following operations:

determining a threshold Thresh_{ADC} which is used to judge whether the ADC values in the region with high DWI values and the region with high DWI values are mismatched according to obtained statistical data on whether N patients are treated with thrombolysis and whether prognoses of the patients are good or bad, wherein N is a natural number greater than 1; and

if the ADCᵣ in the region with high DWI values in the magnetic resonance diffusion weighted imaging is not less than the threshold Thresh_{ADC}, judging that the ADC values in the region with high DWI values and the region with high DWI values are mismatched.

Assuming the foregoing is the fourth possible implementation manner, in a fifth possible implementation manner based on the fourth possible implementation manner, the memory further comprises instructions for performing the following operations:

obtaining a value S_{TP}/(S_{TP}+S_{FN}) indicative of sensitivity and a value S_{TN}/(S_{FP}+S_{TN}) indicative of specificity by performing statistics, among the N patients, on a sum S_{TP} of patients who have good prognosis after thrombolysis and who have bad prognosis without thrombolysis when ADCᵣ is greater than or equal to a threshold Thresh₁ to be determined, a sum S_{TN} of patients who have bad prognosis after thrombolysis and who have good prognosis without thrombolysis when ADCᵣ is less than the threshold Thresh1 to be determined, a sum S_{FP} of patients who have bad prognosis after thrombolysis and who have good prognosis without thrombolysis when ADCᵣ is greater than or equal to the threshold Thresh1 to be determined, and a sum S_{FN} of patients who have good prognosis after thrombolysis and who have bad prognosis without thrombolysis when ADCᵣ is less than the threshold Thresh1 to be determined; and

calculating a value of the threshold Thresh₁ to be determined that makes S_{TP}/(S_{TP}+S_{FN})+S_{TN}/(S_{FP}+S_{TN}) reach a maximum value, and using the value of the threshold Thresh₁ to be determined that makes S_{TP}/(S_{TP}+S_{FN})+S_{TN}/(S_{FP}+S_{TN}) reach the maximum value as the threshold Thresh_{ADC} which is used to judge whether the ADC values in the region with high DWI values and the region with high DWI values are mismatched.

According to another aspect, a further embodiment of the present invention provides a computer-readable storage media, the computer-readable storage media may be included in the memory in the foregoing embodiments or may be a separate one that is not installed in a terminal. The computer-readable storage media store one or more than one program, and the one or more than one program is used by one or more than one processor to execute the method for determining characteristic of cerebral ischemia based on magnetic resonance diffusion weighted imaging, wherein the method comprises:

determining a cerebral ischemia region of a patient based on magnetic resonance diffusion weighted imaging of the patient, wherein the cerebral ischemia region comprises a core region and a transition region;

determining a apparent diffusion coefficient ADC characteristic parameter ADCᵣ in a region with high DWI values in the magnetic resonance diffusion weighted imaging according to diffusion weighted image DWI values in the core region and transition region; and

judging whether ADC values in the region with high DWI values and the region with high DWI values are mismatched according to the ADC characteristic parameter ADCᵣ.

Assuming the foregoing is a first possible implementation manner, in a second possible implementation manner based on the first possible implementation manner, the step of determining a cerebral ischemia region of a patient based on magnetic resonance diffusion weighted imaging of the patient comprises:

calculating the ADC values of voxels in the magnetic resonance diffusion weighted imaging; determining a region of which the ADC values of voxels in the magnetic resonance diffusion weighted imaging are less than D₁×ADC_{ref} as the core region; and determining a region of which the ADC values of voxels in the magnetic resonance diffusion weighted imaging are in the range of [D₁×ADC_{ref}, D₂×ADC_{ref}] and that is spatially adjacent to the core region as the transition region, wherein ADC_{ref} is the ADC value of normal brain tissues, D₁ is a constant in the range of [0.6, 0.7], and D₂ is a constant in the range of [0.8, 0.9].

Assuming the foregoing is the second possible implementation manner, in a third possible implementation manner based on the first possible implementation manner, the step of determining an apparent diffusion coefficient ADC characteristic parameter ADCᵣ in a region with high DWI values in the magnetic resonance diffusion weighted imaging according to diffusion weighted image DWI values of the core region and transition region comprises:

calculating an average DWI gray scale value DWI_{avg} and a maximum DWI value DWIₘₐₓ according to the DWI values in the core region;

determining a region that consists of voxels with the DWI values being not less than Th₁ in the core region and transition region as the region with high DWI values in the magnetic resonance diffusion weighted imaging, wherein Th₁ is a preset value or a constant in the range of (DWI_{avg}+DWIₘₐₓ)/2, 1.1×(DWI_{avg}+DWIₘₐₓ)/2]; and

calculating the ADC values in the region with high DWI values, and using a ratio of the voxels in the region with high DWI values and whose ADC values are not less than C₁×ADC_{ref} to all voxels in the region with high DWI values as the ADC characteristic parameter ADCᵣ in the region with high DWI values in the magnetic resonance diffusion weighted imaging, wherein C1 is a constant in the range of [0.6, 0.7], and ADC_{ref} is the ADC values of normal brain tissues.

Assuming the foregoing is the third possible implementation manner, in a fourth possible implementation manner based on the first possible implementation manner, the step of judging whether ADC values in the region with high DWI values and the region with high DWI values are mismatched according to the ADC characteristic parameter ADCᵣ comprises:

determining a threshold Thresh_{ADC} which is used to judge whether the ADC values in the region with high DWI values and the region with high DWI values are mismatched according to obtained statistical data on whether N patients are treated with thrombolysis and whether prognoses of the patients are good or bad, wherein N is a natural number greater than 1; and

if the ADCᵣ in the region with high DWI values in the magnetic resonance diffusion weighted imaging is not less than the threshold Thresh_{ADC}, judging that the ADC values in the region with high DWI values and the region with high DWI values are mismatched.

Assuming the foregoing is the fourth possible implementation manner, in a fifth possible implementation manner based on the fourth possible implementation manner, the step of determining a threshold Thresh_{ADC} which is used to judge whether the ADC values in the region with high DWI values and the region with high DWI values are mismatched according to obtained statistical data on whether N patients are treated with thrombolysis and whether prognoses of the patients are good or bad comprises:

obtaining a value S_{TP}/(S_{TP}+S_{FN}) indicative of sensitivity and a value S_{TN}/(S_{FP}+S_{TN}) indicative of specificity by performing statistics, among the N patients, on a sum S_{TP} of patients who have good prognosis after thrombolysis and who have bad prognosis without thrombolysis when ADCᵣ is greater than or equal to a threshold Thresh₁ to be determined, a sum S_{TN} of patients who have bad prognosis after thrombolysis and who have good prognosis without thrombolysis when ADCᵣ is less than the threshold Thresh1 to be determined, a sum S_{FP} of patients who have bad prognosis after thrombolysis and who have good prognosis without thrombolysis when ADCᵣ is greater than or equal to the threshold Thresh1 to be determined, and a sum S_{FN} of patients who have good prognosis after thrombolysis and who have bad prognosis without thrombolysis when ADCᵣ is less than the threshold Thresh1 to be determined; and

calculating a value of the threshold Thresh₁ to be determined that makes S_{TP}/(S_{TP}+S_{FN})+S_{TN}/(S_{FP}+S_{TN}) reach a maximum value, and using the value of the threshold Thresh₁ to be determined that makes S_{TP}/(S_{TP}+S_{FN})+S_{TN}/(S_{FP}+S_{TN}) reach the maximum value as the threshold Thresh_{ADC} which is used to judge whether the ADC values in the region with high DWI values and the region with high DWI values are mismatched.

It should be noted that content such as information interaction and execution processes among the modules/units of the foregoing apparatus are based on the same conception as the method embodiment of the present invention, and they have the same technical effects as those described in the method embodiment of the present invention. For details, reference may be made to descriptions of the method embodiment of the present invention, which is not described herein again.

It may be understood by a person of ordinarily skills in the art that, all or part of the steps in the methods of the foregoing embodiments can be executed by a program instructing relevant hardware, and the program can be stored in a computer-readable storage medium. The computer-readable storage medium may comprise: a Read-Only Memory (ROM), a Random Access Memory (RAM), a magnetic disk, or an optical disk, etc.

The above provides a detailed description of the method and apparatus for determining characteristics of cerebral ischemia based on magnetic resonance diffusion weighted imaging according to the embodiments of the present invention, where the specific implementation methods are applied to illustrate the principle and embodiments of the present invention; and the foregoing embodiments are merely for ease of understanding of the method and core ideas of the present invention; meanwhile, for a person of ordinary skill in the art, on the basis of the idea of the present invention, a modification may be made to the specific implementing method and the application range. In conclusion, the content of this specification shall not be construed as a limitation on the present invention.

What is claimed is:

1. A method for determining characteristics of cerebral ischemia based on magnetic resonance diffusion weighted imaging, wherein the method comprises:
determining a cerebral ischemia region of a patient based on magnetic resonance diffusion weighted imaging of the patient, wherein the cerebral ischemia region comprises a core region and a transition region;
determining an apparent diffusion coefficient ADC characteristic parameter ADCᵣ in a region with high DWI values in the magnetic resonance diffusion weighted imaging according to diffusion weighted image DWI values in the core region and transition region; and
judging whether ADC values in the region with high DWI values and the region with high DWI values are mismatched according to the ADC characteristic parameter ADCᵣ.

2. The method of claim 1, wherein the step of determining a cerebral ischemia region of a patient based on magnetic resonance diffusion weighted imaging of the patient comprises:
calculating the ADC values of voxels in the magnetic resonance diffusion weighted imaging; determining a region of which the ADC values of voxels in the magnetic resonance diffusion weighted imaging are less than D₁×ADC_{ref} as the core region; and determining a region of which the ADC values of voxels in the magnetic resonance diffusion weighted imaging are in the range of [D₁×ADC_{ref}, D₂×ADC_{ref}] and that is spatially adjacent to the core region as the transition region, wherein ADC_{ref} is the ADC value of normal brain tissues, D₁ is a constant in the range of [0.6, 0.7], and D₂ is a constant in the range of [0.8, 0.9].

3. The method of claim 1, wherein, the step of determining an apparent diffusion coefficient ADC characteristic parameter ADCᵣ in a region with high DWI values in the magnetic resonance diffusion weighted imaging according to diffusion weighted image DWI values of the core region and transition region comprises:
calculating an average DWI gray scale value DWI_{avg} and a maximum DWI value DWIₘₐₓ according to the DWI values in the core region;
determining a region that consists of voxels with the DWI values being not less than Th₁ in the core region and transition region as the region with high DWI values in the magnetic resonance diffusion weighted imaging, wherein Th₁ is a preset value or a constant in the range of (DWI_{avg}+DWIₘₐₓ)/2, 1.1×(DWI_{avg}+DWIₘₐₓ)/2]; and
calculating the ADC values in the region with high DWI values, and using a ratio of the voxels in the region with high DWI values and whose ADC values are not less than C₁×ADC_{ref} to all voxels in the region with high DWI values as the ADC characteristic parameter ADCᵣ in the region with high DWI values in the magnetic resonance diffusion weighted imaging, wherein C1 is a constant in the range of [0.6, 0.7], and ADC_{ref} is the ADC values of normal brain tissues.

4. The method of claim 1, wherein, the step of judging whether ADC values in the region with high DWI values and the region with high DWI values are mismatched according to the ADC characteristic parameter ADCᵣ comprises:
determining a threshold Thresh_{ADC} which is used to judge whether the ADC values in the region with high DWI values and the region with high DWI values are mismatched according to obtained statistical data on whether N patients are treated with thrombolysis and whether prognoses of the patients are good or bad, wherein N is a natural number greater than 1; and
if the ADCᵣ in the region with high DWI values in the magnetic resonance diffusion weighted imaging is not less than the threshold Thresh_{ADC}, judging that the ADC values in the region with high DWI values and the region with high DWI values are mismatched.

5. The method of claim 4, wherein, the step of determining a threshold Thresh_{ADC} which is used to judge whether the ADC values in the region with high DWI values and the region with high DWI values are mismatched according to obtained statistical data on whether N patients are treated with thrombolysis and whether prognoses of the patients are good or bad comprises:
obtaining a value S_{TP}(S_{TP}+S_{FN}) indicative of sensitivity and a value S_{TN}/(S_{FP}+S_{TN}) indicative of specificity by performing statistics, among the N patients, on a sum S_{TP} of patients who have good prognosis after thrombolysis and who have bad prognosis without thrombolysis when ADCᵣ is greater than or equal to a threshold Thresh₁ to be determined, a sum S_{TN} of patients who have bad prognosis after thrombolysis and who have good prognosis without thrombolysis when ADCᵣ is less than the threshold Thresh1 to be determined, a sum S_{FP} of patients who have bad prognosis after thrombolysis and who have good prognosis without thrombolysis when ADCᵣ is greater than or equal to the threshold Thresh1 to be determined, and a sum S_{FN} of patients who have good prognosis after thrombolysis and who have bad prognosis without thrombolysis when ADCᵣ is less than the threshold Thresh1 to be determined; and
calculating a value of the threshold Thresh₁ to be determined that makes S_{TP}/(S_{TP}+S_{FN})+S_{TN}/(S_{FP}+S_{TN}) reach a maximum value, and using the value of the threshold Thresh₁ to be determined that makes S_{TP}/(S_{TP}+S_{FN})+S_{TN}/(S_{FP}+S_{TN}) reach the maximum value as the threshold Thresh_{ADC} which is used to judge whether the ADC values in the region with high DWI values and the region with high DWI values are mismatched.

6. An apparatus for determining characteristics of cerebral ischemia based on magnetic resonance diffusion weighted imaging, wherein the apparatus comprises:
a cerebral ischemia region determining module, configured to determine a cerebral ischemia region of a patient based on magnetic resonance diffusion weighted imaging DWI of the patient, wherein the cerebral ischemia region comprises a core region and a transition region;
a characteristic parameter determining module, configured to determine an apparent diffusion coefficient ADC characteristic parameter ADCᵣ in a region with high DWI values in the magnetic resonance diffusion weighted imaging according to DWI values in the core region and transition region; and
a judging module, configured to judge whether ADC values in the region with high DWI values and the region with high DWI values are mismatched according to the ADC characteristic parameter ADCᵣ.

7. The apparatus of claim 6, wherein the cerebral ischemia region determining module comprises:
a first calculating unit, configured to calculate the ADC values of voxels in the magnetic resonance diffusion weighted imaging; determine a region of which the ADC values of voxels in the magnetic resonance diffusion weighted imaging are less than D₁×ADC_{ref} as the core region; and determine a region of which the ADC values of voxels in the magnetic resonance diffusion weighted imaging are in the range of [D₁×ADC_{ref}, D₂×ADC_{ref}] and that is spatially adjacent to the core region as the transition region, wherein ADC_{ref} is the ADC values of normal brain tissues, D₁ is a constant in the range of [0.6, 0.7], and D₂ is a constant in the range of [0.8,0.9].

8. The apparatus of claim 6, wherein the characteristic parameter determining module comprises:
a second calculating unit, configured to calculate an average DWI gray scale value DWI_{avg} and a maximum DWI value DWIₘₐₓ according to the DWI values in the core region;
a first determining unit, configured to determine a region that consists of the voxels with the DWI values being not less than Th₁ in the core region and transition region as the region with high DWI values in the magnetic resonance diffusion weighted imaging, wherein Th₁ is a preset value or a constant in the range of (DWI_{avg}+DWIₘₐₓ)/2, 1.1×(DWI_{avg}+DWIₘₐₓ)/2]; and
a second determining unit, configured to calculate the ADC values in the region with high DWI values, and use a ratio of the voxels in the region with high DWI values and whose ADC values are not less than C₁×ADC_{ref} to all voxels in the region with high DWI values as the ADC characteristic parameter ADCᵣ in the region with high DWI values in the magnetic resonance diffusion weighted imaging, wherein C1 is a constant in the range of [0.6, 0.7], and ADC_{ref} is the ADC values of normal brain tissues.

9. The apparatus of claim 6, wherein the judging module comprises:
a third determining submodule, configured to determine a threshold Thresh_{ADC} which is used to judge judging whether the ADC values in the region with high DWI values and the region with high DWI values are mismatched according to obtained statistical data on whether N patients are treated with thrombolysis and whether prognoses of the patients are good or bad, wherein N is a natural number greater than 1; and
a first judging submodule, configured to, if the ADCᵣ in the region with high DWI values in the magnetic resonance diffusion weighted imaging is not less than the threshold Thresh_{ADC}, judge that the ADC values in the region with high DWI values and the region with high DWI values are mismatched.

10. The apparatus of claim 9, wherein the third determining submodule comprises:
a statistics collecting unit, configured to obtain a value S_{TP}(S_{TP}+S_{FN}) indicative of sensitivity and a value S_{TN}/(S_{FP}+S_{TN}) indicative of specificity by performing statistics, among the N patients, on a sum S_{TP} of patients who have good prognosis after thrombolysis and who have bad prognosis without thrombolysis when ADCᵣ is greater than or equal to a threshold Thresh1 to be determined, a sum S_{TN} of patients who have bad prognosis after thrombolysis and who have good prognosis without thrombolysis when ADCᵣ is less than the threshold Thresh1 to be determined, a sum S_{FP} of patients who have bad prognosis after thrombolysis and who have good prognosis without thrombolysis s when ADCᵣ is greater than or equal to the threshold Thresh1 to be determined, and a sum S_{FN} of patients who have good prognosis after thrombolysis and who have bad prognosis without thrombolysis when ADCᵣ is less than the threshold Thresh1 to be determined; and
an obtaining unit, configured to calculate a value of the threshold Thresh1 to be determined that makes S_{TP}/(S_{TP}+S_{FN})+S_{TN}/(S_{FP}+S_{TN}) reach a maximum value, and use the value of the threshold Thresh1 to be determined that makes S_{TP}/(S_{TP}+S_{FN})+S_{TN}/(S_{FP}+S_{TN}) reach the maximum value as the threshold Thresh_{ADC} which is used to judge whether the ADC values and the region with high DWI values are mismatched.
